(19) United States
(12) Patent Application Publication
(10) Pub. No.: US 2014/0024986 A1
(43) Pub. Date: Jan. 23, 2014

(54) LIMB COMPRESSION DEVICE AND CONTROL METHOD

(71) Applicant: Terumo Kabushiki Kaisha, Shibuya-ku (JP)

(72) Inventor: Takahiro Souma, Ashigarakami-gun (JP)

(73) Assignee: Terumo Kabushiki Kaisha, Shibuya-ku (JP)

(21) Appl. No.: 14/035,629

(22) Filed: Sep. 24, 2013

Related U.S. Application Data
(63) Continuation of application No. PCT/JP2012/001684, filed on Mar. 12, 2012.

(30) Foreign Application Priority Data
Mar. 31, 2011 (JP) 2011-078016

(51) Int. Cl. A61H 9/00; A61B 5/021

(52) U.S. Cl. CPC A61H 9/0078; A61B 5/021
USPC 601/149

(57) ABSTRACT

A limb compression device to which a cuff unit wound around a limb of a patient is connected controls compression and release of the limb by controlling air supply and exhaust of the cuff unit to repeat an compression period and a reperfusion period a predetermined number of times. At the start of the compression period, pressurization using the cuff unit is performed up to a compression pressure value based on a systolic blood pressure of the patient measured by detection of pulsation. During the compression period, the device repeats depressurization of the cuff unit at a low rate until pulsation is detected and pressurization of the cuff unit after the depressurization so as to eliminate pulsation.

This application is a continuation of International Patent Application No. PCT/JP2012/001684 filed on Mar. 12, 2012, and claims priority to Japanese Patent Application No. 2011-078016 filed on Mar. 31, 2011, the entire content of both of which is incorporated herein by reference.

### BACKGROUND OF THE INVENTION

1. Field of the Invention

The present invention relates to a limb compression device and control method thereof, which repeats a compression state by cuff (occlusion state by cuff) and a reperfusion state (release state by cuff) by controlling the compression state and release state of the upper limb and/or lower limb.

2. Description of the Related Art

In medical treatment for an ischemic disease, it is an indispensable subject to reduce the influence of myocardial ischemia on myocardial cells. Early re-perfusion is important to protect the cardiac muscle. In general, a treatment using a thrombolytic agent is performed. In addition, recently, there has been performed a treatment by PTCA, that is, performing angiography and re-perfusion by using a catheter while observing an ischemic region.

These treatments for the implementation of early re-perfusion have great effects, but inflict large damage to the cardiac muscle. More specifically, re-perfusion causes trouble or inflammation to the myocardial cells, resulting in the enlargement of an ischemic region (re-perfusion syndrome). This poses a great challenge on medical treatments.

The cause of this is active oxygen produced by myocardial cells. It is important for the prevention of a re-perfusion syndrome to suppress this production.

In order to achieve this challenge, RICM (Remote Ischemic Conditioning method) has been proposed, which suppresses the production of active oxygen which inflicts damage to the cardiac muscle as an ischemic region by setting a region spaced apart from the ischemic region, for example, the upper arm or lower limb in a compression state and a release state and repeating a compression state by cuff/reperfusion state by cuff before re-perfusion of blood to the cardiac muscle.

RICM can suppress the induction of kinase which is an enzyme contributing to the production of active oxygen upon occurrence of intracellular or intercellular mutation of an intracellular intermediate substance by repetitive stimulation. It is possible to suppress cell activity by suppressing the metabolism of ATP of myocardial cells and adjusting the function of a mitochondria in the myocardial cells, thereby suppressing the production of active oxygen.

Conventionally, various types of limb compression devices have been proposed as devices for implementing RICM to suppress such a re-perfusion syndrome. For example, PTL 1 discloses an arrangement which can arbitrarily set a compression (occlusion) period by cuff, a reperfusion period by cuff, a compression pressure, and the like, and also describes that it is possible to control the compression pressure.

### CITATION LIST

### Patent Literature

PTL 1: Japanese PCT National Publication 2010-512176

On the other hand, when performing treatment by RICM, since it imposes a heavy burden on the patient, it is necessary to set a compression period, reperfusion period, compression (occlusion) pressure, and the like so as to minimize the burden on the patient in consideration of the severity of the patient's illness. Minimizing the compression pressure instead of unnecessarily increasing it, in particular, will greatly contribute to a reduction in burden on the patient. This makes it necessary to use a limb compression device which can control pressure with very high precision.

Although PTL 1 discloses the arrangement which can make settings so as to minimize the burden on the patient and sets a compression pressure by cuff higher than a systolic blood pressure by a specified pressure, there is no description about any concrete arrangement for implementing limb compressing operation suitable for variations in systolic blood pressure. In addition, although this technique can cope with increases in the blood pressure value of a patient, it cannot cope with decreases in the blood pressure value of the patient. In this case, it is not possible to reduce the burden on the patient.

In consideration of the above problem, one embodiment of the present invention provides a limb compression device which can fully automatically perform proper operation in an emergency state and minimize burden on a patient and implement limb compressing operation with a proper pressure.

### SUMMARY OF THE INVENTION

According to one aspect of the present invention, there is provided a limb compression device to which a cuff unit wound around a limb of a patient is connected and which controls compression and release of the limb by controlling air supply and exhaust of the cuff unit, the limb compression device comprising: a compression unit configured to compress the limb over a predetermined compression period (ischemic period) by controlling air supply and exhaust of the cuff unit; a release unit configured to release the compression of the limb over a predetermined reperfusion period by controlling exhaust of the cuff unit; a control unit configured to repeat the ischemic period by the compression unit and the reperfusion period by the release unit by a predetermined number of times; and a detection unit configured to detect pulsation on a peripheral side relative to a region compressed by the cuff unit, wherein the compression unit performs pressurization using the cuff unit up to a compression pressure value based on a systolic blood pressure of the patient measured by detection of pulsation using the detection unit, and repeats, in the compression period, depressurization of the cuff unit at a low rate until pulsation is detected by the detection unit and pressurization of the cuff unit after the depressurization so as to eliminate pulsation.

Other features and advantages of the present invention will be apparent from the following description taken in conjunction with the accompanying drawings. Note that the same reference numerals denote the same or like components throughout the accompanying drawings.

### BRIEF DESCRIPTION OF THE DRAWINGS

The accompanying drawings, which are incorporated in and constitute a part of the specification, illustrate exemplary embodiments of the invention and, together with the description, serve to explain the principles of the present invention.

FIG. 1 is a view showing the outer arrangement of a limb compression device 100 according to an embodiment of the present invention;

FIG. 2 is a view showing an example of the display window displayed on the display unit of the limb compression device 100;

FIG. 3 is a view showing how a patient is treated by the remote ischemic conditioning method using the limb compression device 100;

FIG. 4 is a block diagram showing the functional arrangement of the limb compression device 100;

FIG. 5A is a flowchart showing a procedure for remote ischemic conditioning processing in the limb compression device 100;

FIG. 5B is a flowchart showing a procedure for remote ischemic conditioning processing in the limb compression device 100;

FIG. 6 is a flowchart showing a procedure for remote ischemic conditioning processing in the limb compression device 100;

FIG. 7 is a flowchart showing a procedure for remote ischemic conditioning processing in the limb compression device 100;

FIGS. 8A and 8B are flowcharts showing procedures for remote ischemic conditioning processing in the limb compression device 100; and

FIG. 9 is a graph for explaining pressure control by the limb compression device 100.

### DESCRIPTION OF THE EMBODIMENTS

The preferred embodiments of the present invention will be described below with reference to the accompanying drawings.

### First Embodiment

### 1. Outer Arrangement of Limb Compression Device

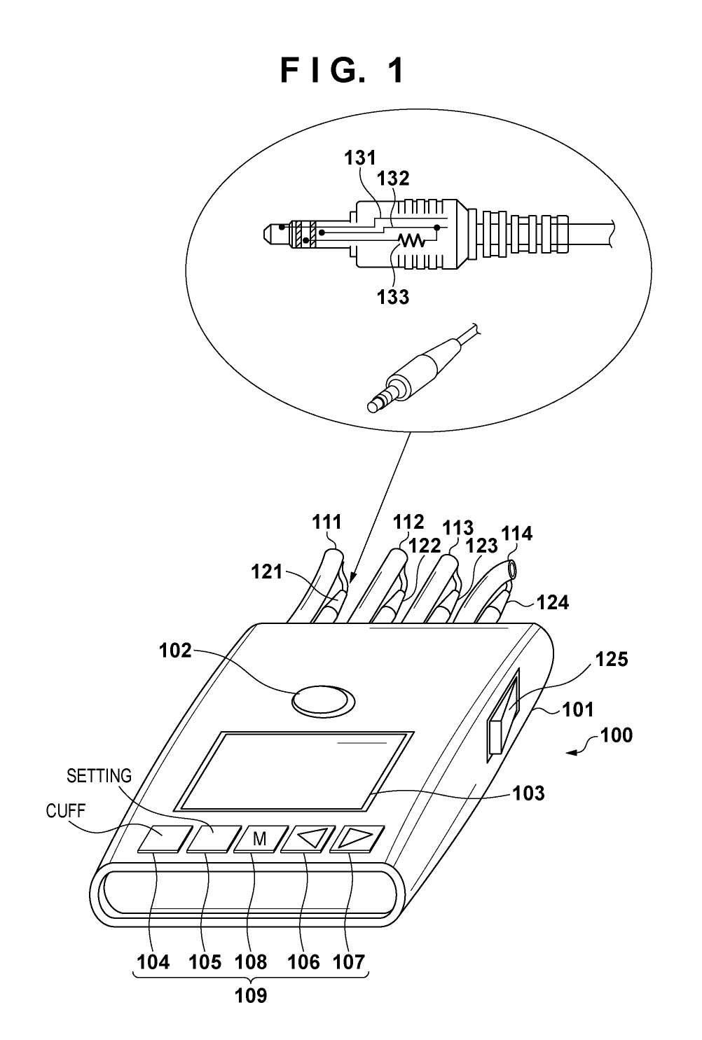

The outer arrangement of a limb compression device 100 according to an embodiment of the present invention will now be described. FIG. 1 shows the outer arrangement of the limb compression device 100 according to an embodiment of the present invention.

Referring to FIG. 1, reference numeral 101 denotes a housing having a side surface provided with air connectors (not shown) for the connection of tubes 111 to 114 attached with cuff units (not shown) which are respectively wound around the right lower limb, left lower limb, right upper arm, and left upper arm and microphone connectors 121 to 124 connected to K-sound microphones (not shown) arranged on the peripheral sides of the four limbs on the surface sides of the air bladders (not shown) of the cuffs which are in contact with the living body at the time of attachment of the cuffs. The microphone connectors 121 to 124 each incorporate signal lines 131 and 132 (the signal line 132 is a ground line) from the microphone and a resistor 133 whose resistance value is set for each cuff so as to discriminate the cuff size. One terminal of the resistor 133 is connected to the signal line 132. The other terminal of the resistor 133 is connected to a predetermined portion of a connector pin. For example, in this embodiment, three types of cuff sizes, namely S, M, and L sizes, are prepared, and the resistors 133 respectively have resistance values of 1 KΩ, 10 KΩ, and 100 KΩ respectively corresponding to S, M, and L sizes. Although described later, the respective cuff units, the connection sensors, tubes communicating with cuffs, and K-sound measurement units which are respectively connected to the cuff units, pressure detection units, and pressure control units respectively constitute cuff systems which independently operate. This embodiment is provided with four cuff systems. Note that it is not always necessary to provide four cuff systems.

Reference numeral 125 denotes a power switch which turns on and off the power supply of the device; 102, a switch which issues an instruction to start/stop remote ischemic conditioning processing (to be referred to as RICM processing hereinafter), and is configured to start processing when being pressed once and to stop processing when being pressed again; and 103, a display unit which displays various types of setting values for the execution of RICM processing, a currently executed state, and the like. Note that the display window displayed on the display unit 103 will be described in detail later.

Reference numeral 109 denotes an operation input unit which includes switches for display switching among pieces of information to a plurality of cuff systems (tubes 111 to 114 which are communicating with cuffs), setting a compression period (an occlusion period)/reperfusion period (non-compression period) which is the length of a compression period or reperfusion period concerning each cuff system, and manually setting a compression pressure. A cuff changeover switch 104 is a switch for changeover operation to display information belonging to a specific one of the four cuff systems. When, for example, the user turns on a setting switch 105, the device is set in a setting mode. The display unit 103 displays abnormality information, cuff pressure value, and compression pressure value concerning the cuff system corresponding to the tube 111. Every time the user presses the cuff changeover switch 104, pieces of abnormality information, cuff pressure values, and compression pressure values concerning the respective cuffs change over like the cuff system corresponding to the tube 112→the cuff system corresponding to the tube 113→the cuff system corresponding to the tube 114→the cuff system corresponding to the tube 111. In this case, compression is a state in which no blood flows because of pressing by a cuff or the like with a predetermined pressure or more.

The setting switch 105 is a switch to be used to sequentially change over setting items like compression period→reperfusion period→compression pressure→cycle count→compression period. A manual mode switch 108 is a switch for changing over to a manual mode to be used when no K-sound can be detected. When the user sets to the manual mode by pressing the manual mode switch 108, the device changes to the manual mode and turns on a manual mode indication. Every time the user presses the manual mode switch 108, the modes change over in the order of automatic mode→manual mode→automatic mode. Setting value increasing/decreasing switches 106 and 107 are switches to be used to increase/decrease the setting value of the item (compression period/reperfusion period/compression pressure/cycle count) selected by the setting changeover switch 105.

### 2. Arrangement of Display Window

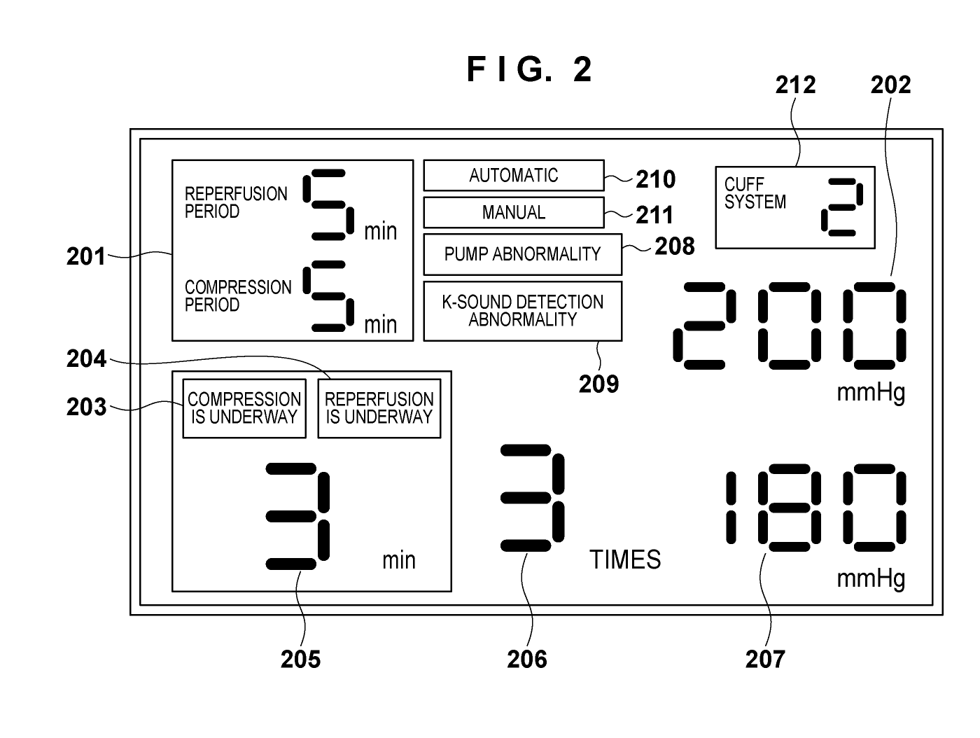

An example of the display window displayed on the display unit 103 will be described next. FIG. 2 is a view showing an example of the display window displayed on the display unit 103. This display window shows abnormality information (pump abnormality and K-sound detection abnormality), cuff pressure value, and compression pressure value concerning one cuff system selected by the cuff changeover switch 104. Therefore, the abnormality information indication, cuff pressure value, and compression pressure value shown in FIG. 2 are independently set for each of the four cuff systems.

Referring to FIG. 2, reference numeral 201 denotes a display area which displays a set compression period and reperfusion period; 202, a display area for displaying a set compression pressure; 203, a display area which displays that compression is underway and is turned on during the occurrence of compression (this area will be referred to as the compression underway indication 203 hereinafter); 204, a display area which indicates that reperfusion is underway and is turned on during reperfusion (this area will be referred to as the reperfusion underway indication 204 hereinafter); 205, a residual time indication which displays a residual time until an ischemic period if compression is underway and displays a residual time until the completion of a reperfusion period if reperfusion is underway; 210, an indication (automatic mode indication) indicating that the device operates in the automatic setting mode; and 211, an indication (manual mode indication) indicating that the device operates in the manual setting mode.

Reference numeral 206 denotes a residual cycle count display area which displays a residual cycle count. Note that the limb compression device according to this embodiment is configured to operate in four cycles, with one cycle including compression and reperfusion, and then automatically stop. A cycle count is a predetermined number of times of iterations in RICM, which is “four” in the following description. As described above, however, the user can set this count by operating the operation input unit 109.

Reference numeral 207 denotes a cuff pressure indication displaying a current cuff pressure value. Note that four types of cuff units for the right lower limb, left lower limb, right upper arm, and left upper arm are connected to the device. As described above, however, it is possible to select to display the cuff pressure value of a specific one of the cuff units by changing over cuff systems as display targets by operating the cuff changeover switch 104. A display area 212 displays the number or an attachment portion name such as the right upper arm, left upper arm, right thigh, or left thigh of the cuff system whose information is currently displayed.

A pump abnormality indication 208 blinks upon detection of air leakage or a trouble in the limb compression device 100 to notify the user of the corresponding information. A K-sound detection abnormality indication 209 blinks, when the limb compression device 100 detects an abnormality concerning the K-sound detection function, including lower sound level because of a shift of the K-sound microphone from the arterial position of each of the four limbs, to notify the user of the corresponding information.

### 3. Use Form of Limb Compression Device

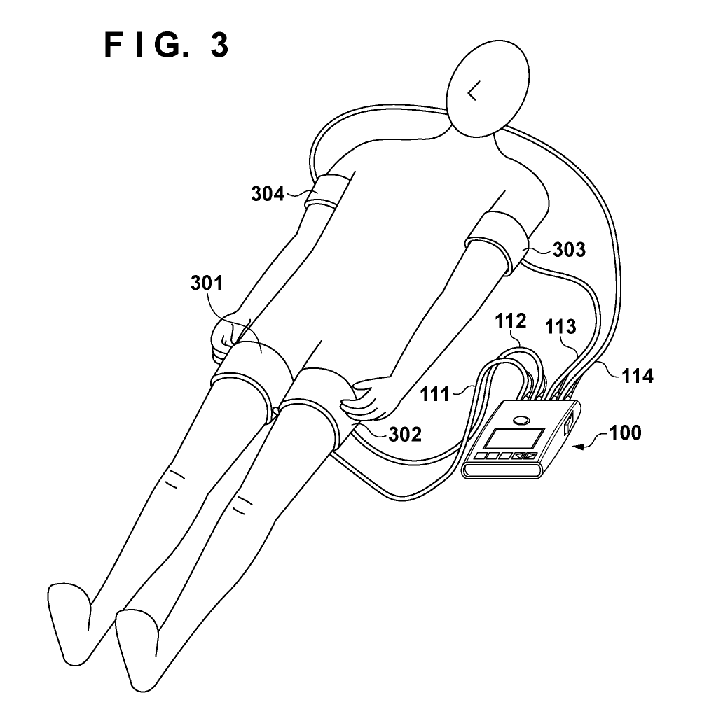

A use form of the limb compression device 100 will be described next. FIG. 3 shows how a patient is treated by the remote ischemic conditioning method using the limb compression device 100.

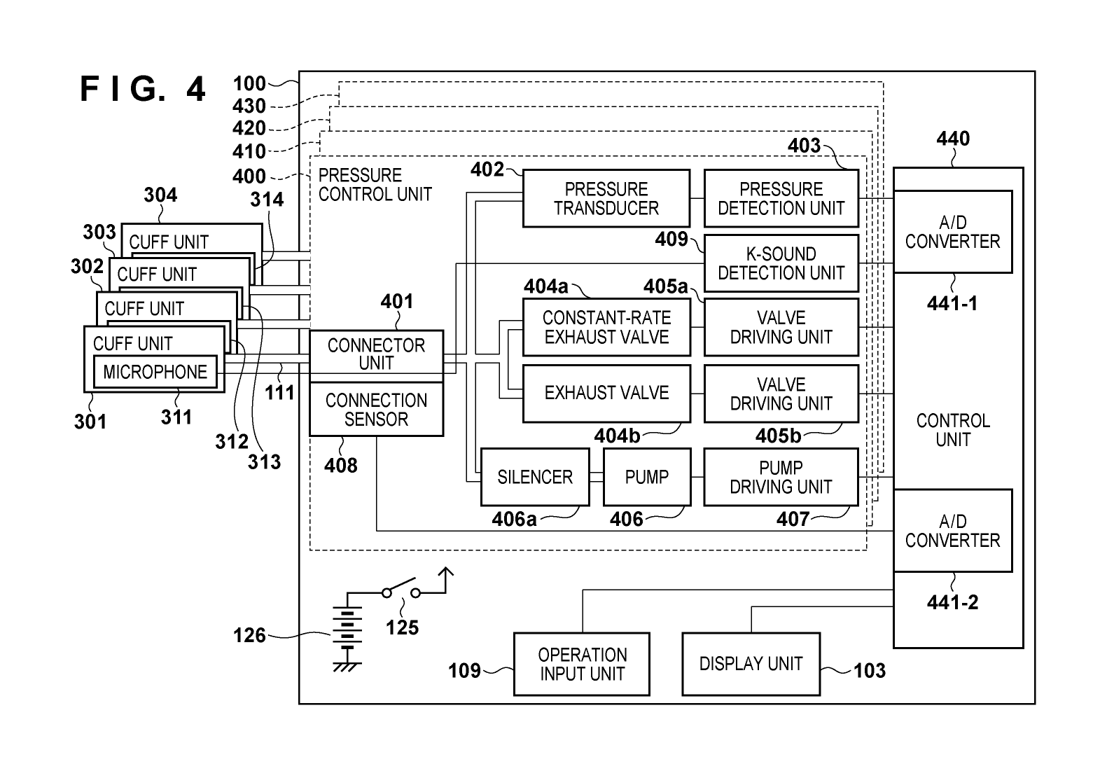

As shown in FIG. 3, a cuff unit 301 for the right lower limb is attached to the distal end of the tube 111. In the case shown in FIG. 3, the cuff unit 301 is attached to the right thigh such that a K-sound detection microphone 311 provided on the cuff unit 301 is located on the artery and located on the peripheral side. A cuff unit 302 for the left lower limb is attached to the distal end of the tube 112. In the case shown in FIG. 3, the cuff unit 302 is attached to the left thigh such that a K-sound detection microphone provided on the cuff unit 302 is located on the artery and located on the peripheral side. Likewise, a cuff unit 303 for the left upper arm is attached to the distal end of the tube 113. The cuff unit 303 is attached to the left upper arm of the patient such that a K-sound detection microphone provided on the cuff unit 303 is located on the artery and located on a hand side (the peripheral side). A cuff unit 304 for the right upper arm is attached to the distal end of the tube 114. The cuff unit 304 is attached to the right upper arm such that a K-sound detection microphone provided on the cuff unit 304 is located on the artery and located on a hand side (the peripheral side). Note that the tubes 111 to 114 are also provided with signal lines to the K-sound detection microphones. One end of each of the tubes 111 to 114 is connected to a corresponding one of K-sound detection microphones 311, 312, 313, and 314 (FIG. 4). Each of the microphone connectors 121, 122, 123, and 124 is connected to the other end of a corresponding one of the tubes 111 to 114. The connectors 121 to 124 to which the tubes 111 to 114 and signal lines are connected are preferably detachably attached to connector units 401 and connection sensors 408 of pressure control units 400, 410, 420, and 430 (FIG. 4), respectively.

As described above, the limb compression device 100 according to this embodiment compresses the four limbs of a patient with air pressure by using the cuff units 301 to 304 so as to set the peripheral sides from the respective compressed regions in an ischemic state.

### 4. Functional Arrangement of Limb Compression Device

The functional arrangement of the limb compression device 100 will be described next. FIG. 4 is a block diagram showing the functional arrangement of the limb compression device 100. As shown in FIG. 4, the limb compression device 100 includes the pressure control units 400, 410, 420, and 430, a power supply battery 126, the power switch 125, a control unit 440, the operation input unit 109, and the display unit 103. The control unit 440 includes an A/D converter 441-1 having eight or more input channels, an A/D converter 441-2 having four or more input channels, a CPU such as a microcomputer, a ROM storing control programs for the overall device which are executed by the CPU and various types of data, and a RAM which serves as a work area and temporarily stores measured data and various types of data. The A/D converter 441-1 converts analog signals from a pressure detection unit 403 and a K-sound detection unit 409 into digital signals which can be processed by the CPU. The A/D converter 441-2 converts analog signals from the connection sensor 408 into digital signals which can be processed by the CPU. Although the above description has exemplified the case in which one control unit 440 controls the four pressure control units 400 to 430, this device may use a control scheme of providing a dedicated control unit for each pressure control unit.

The pressure control units 400 to 430 are provided by the number of cuff units 301 to 304 as pressure control targets. The cuff units, the cuff tubes, and the pressure control units respectively constitute the independent cuff systems. That is, the pressure control units 400 to 430 are configured to individually perform pressure control for each cuff unit based on an instruction from the control unit 440 which performs processing/determination in each processing procedure. Note that the four cuff systems have the same arrangement, and hence the pressure control units 400 to 430 have the same arrangement. The pressure control unit 400 which performs pressure control of the cuff unit 301 will therefore be described below.

In the pressure control unit 400, reference numeral 401 denotes a connector unit, to which a connector (not shown) at the proximal end of the tube 111 is detachably connected, wherein the cuff unit 301 is attached to the distal end of the tube 111. Reference numeral 408 denotes a connection sensor which detects the resistance value of the resistor 133 (referring to FIG. 1, for example, S size: 1 kΩ, M size: 10 kΩ, and L size: 100 kΩ) which is mounted in the connector and has a resistance value determined for each cuff size when the connector of the tube 111 is connected, and transmits the resistance value to the control unit 440. For example, the connection sensor 408 supplies a constant current to the resistor 133 indicating a cuff size which is built in the connector of each cuff shown in FIG. 1, converts the current into a voltage value between a resistor R and a negative (ground) electrode, and supplies the voltage value to the A/D converter 441-2 having four or more input channels, thereby detecting a cuff size. The control unit 440 detects the size of a connected cuff by using the connection sensor 408, and inhibits the execution of remote ischemic conditioning processing (to be described later) for a cuff system about which the control unit detects an infinite resistance value and determines that the cuff unit is not connected. Note that each cuff unit includes a detector for detecting pulsation on the peripheral side relative to the region compressed by the cuff unit and a microphone (MIC) for acquiring Korotkoff sound (to be also referred to as K-sound hereinafter) is provided as the detector. The signal line of the microphone 311 of the cuff unit 301 is connected to the K-sound detection unit 409 of the pressure control unit 400 via the connector unit 401. The K-sound detection unit 409 detects K-sound from the sound (signal) acquired by the microphone 311, and notifies the control unit 440 of the corresponding information. Although this embodiment uses K-sound to detect pulsation on the peripheral side relative to the region compressed by the cuff unit 301, the present invention is not limited to this. It is possible to use any known technique using ultrasonic waves, infrared light, or the like.

Reference numeral 402 denotes a pressure transducer connected to the connector unit 401 via a pneumatic pipe. The pressure transducer 402 detects the internal pressure in the cuff unit 301 and converts it into an electrical signal. Reference numeral 403 denotes a pressure detection unit which amplifies the electrical signal output from the pressure transducer 402 and transmits the signal as a cuff pressure signal (pressure value P) to the control unit 440.

Reference numeral 404a denotes a constant-rate exhaust valve as an exhaust means which is connected to the connector unit 401 via a pneumatic pipe. The constant-rate exhaust valve 404a is used to reduce the pressure of the cuff unit 301 at a very low constant-rate during compression and is used to perform depressurization at a specified deflation rate during blood pressure measuring operation. Reference numeral 404b denotes an exhaust valve which serves as a quick exhaust means and is connected to the connector unit 401 via a pneumatic pipe like the constant-rate exhaust valve 404a. The exhaust valve 404b is used to quickly exhaust air from the cuff unit 301 so as to change over the peripheral side relative to the compressed region from a compression state (occlusion state) to a reperfusion state (non-compression state) in which blood is reperfused, and is used for quick air exhaust at the end of blood pressure measurement. That is, the pressure control unit 400 is provided with two types of exhaust valves, namely the exhaust valve 404b having a large exhaust amount per unit time to large decreased cuff pressure and the exhaust valve 404a which controls the exhaust amount to a small constant rate of decreased cuff pressure, which are selectively used in accordance with the processing state of remote ischemic conditioning processing. A valve driving unit 405a controls the orifice area of the constant-rate exhaust valve 404a based on an instruction from the control unit 440. A valve driving unit 405b controls opening/closing of the exhaust valve's 404b orifice based on an instruction from the control unit 440.

Reference numeral 406 denotes a pump which is connected to the connector unit 401 through a silencer 406a and a pneumatic pipe and supplies air to the cuff bag in the cuff unit 301; and 407, a pump driving unit which controls the driving/stopping and pressurization rate of the pump 406 based on an instruction from the control unit 440. The silencer 406a reduces driving sound and pulsation of the pump 406 to prevent the microphone 311 from erroneously detecting the driving sound and pulsation of the pump as K-sound.

The control unit 440 stores programs for executing remote ischemic conditioning processing (to be described later) and controls the valve driving units 405a and 405b, the pump driving unit 407, the display unit 103, and the like based on instructions from the operation input unit 109 including the cuff changeover switch 104, the setting switch 105, the setting value increasing/decreasing switches 106 and 107, and the manual switch 108 and outputs from the pressure detection unit 403.

### 5. Procedure for Remote Ischemic Conditioning Processing (RICM Processing)

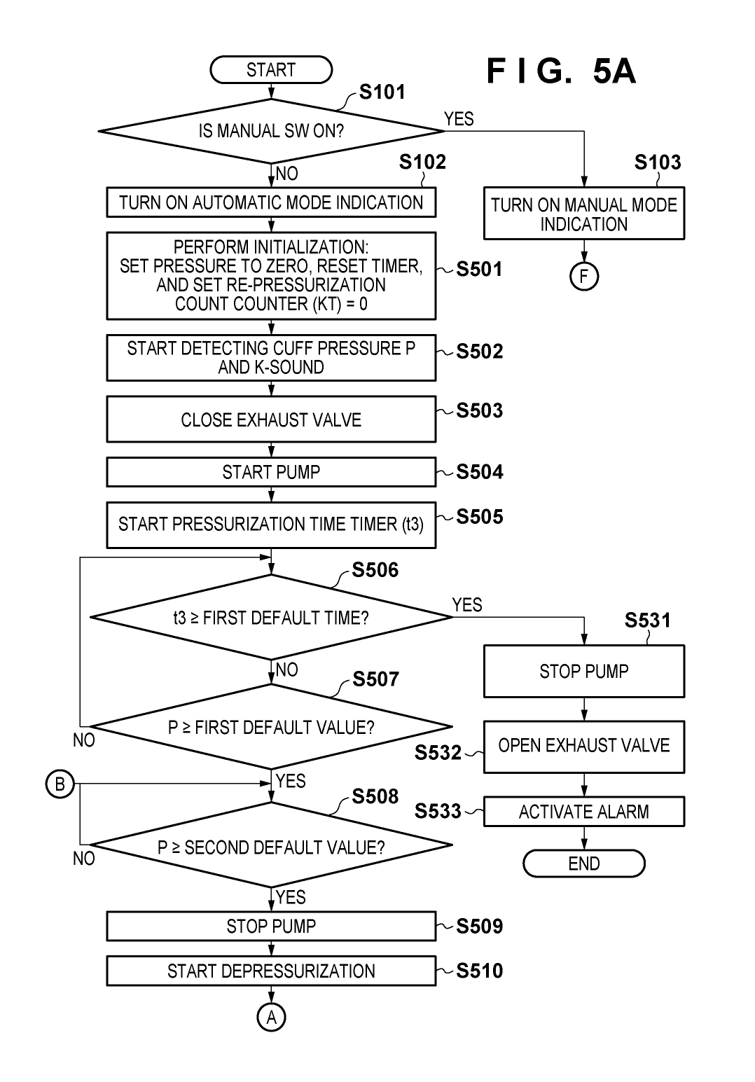

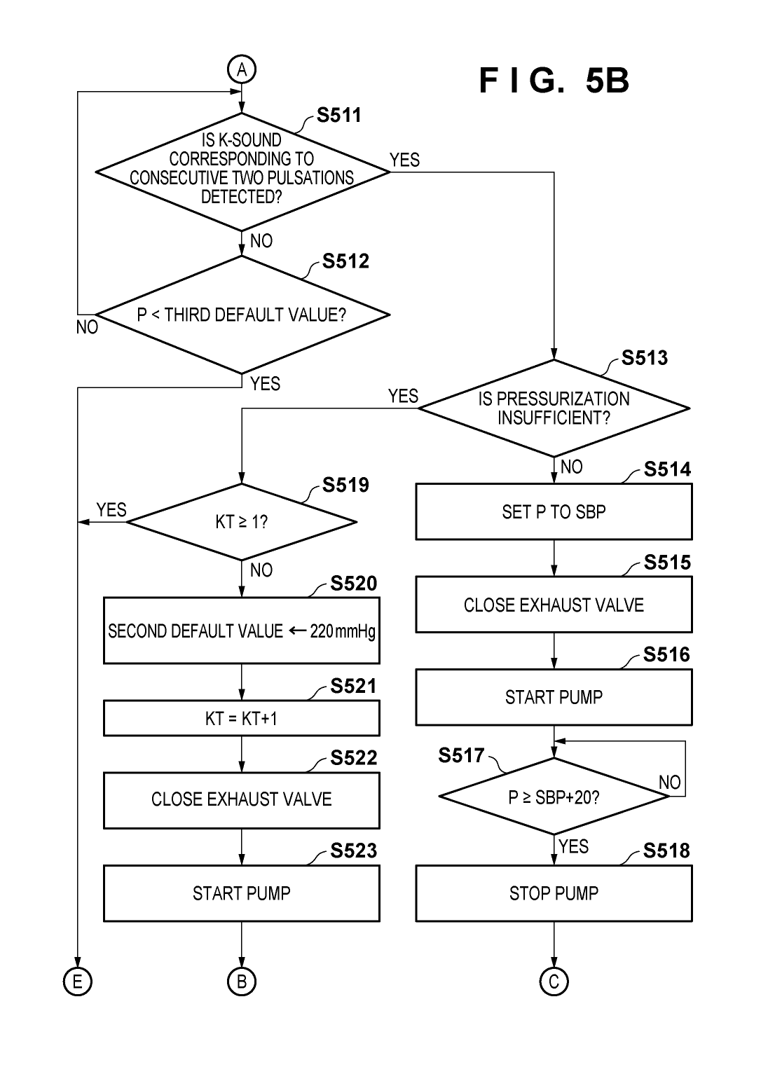

A procedure for RICM processing executed by the control unit 440 of the limb compression device 100 will be described next. FIGS. 5A, 5B, 6, 7, 8A, and 8B are flowcharts showing procedures for RICM processing executed by the control unit 440 of the limb compression device 100.

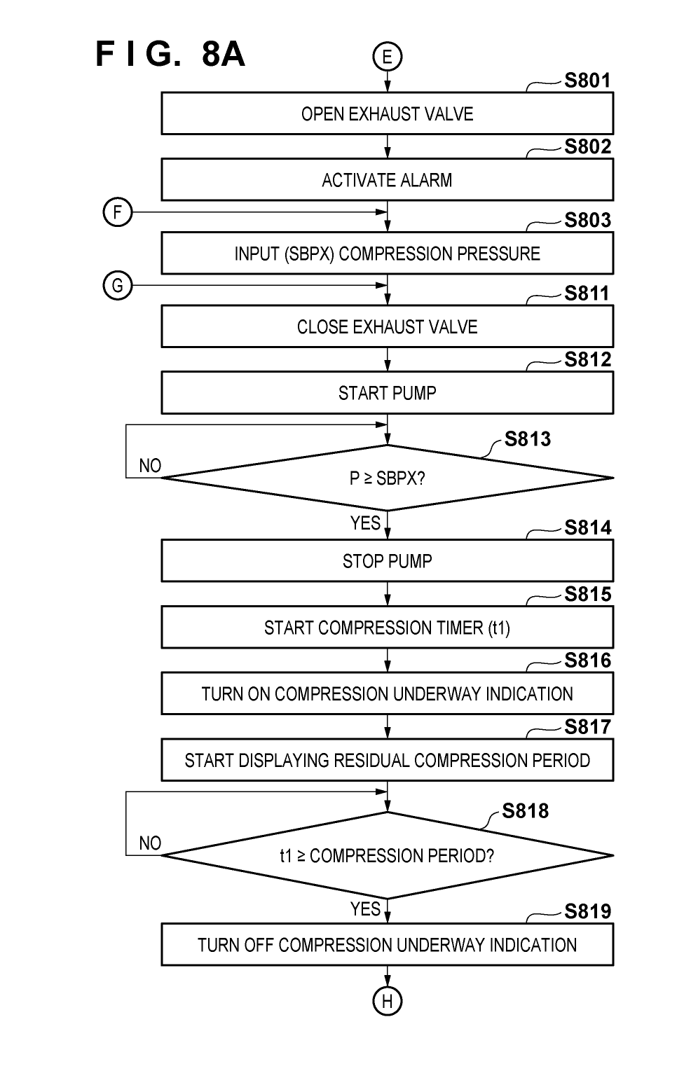

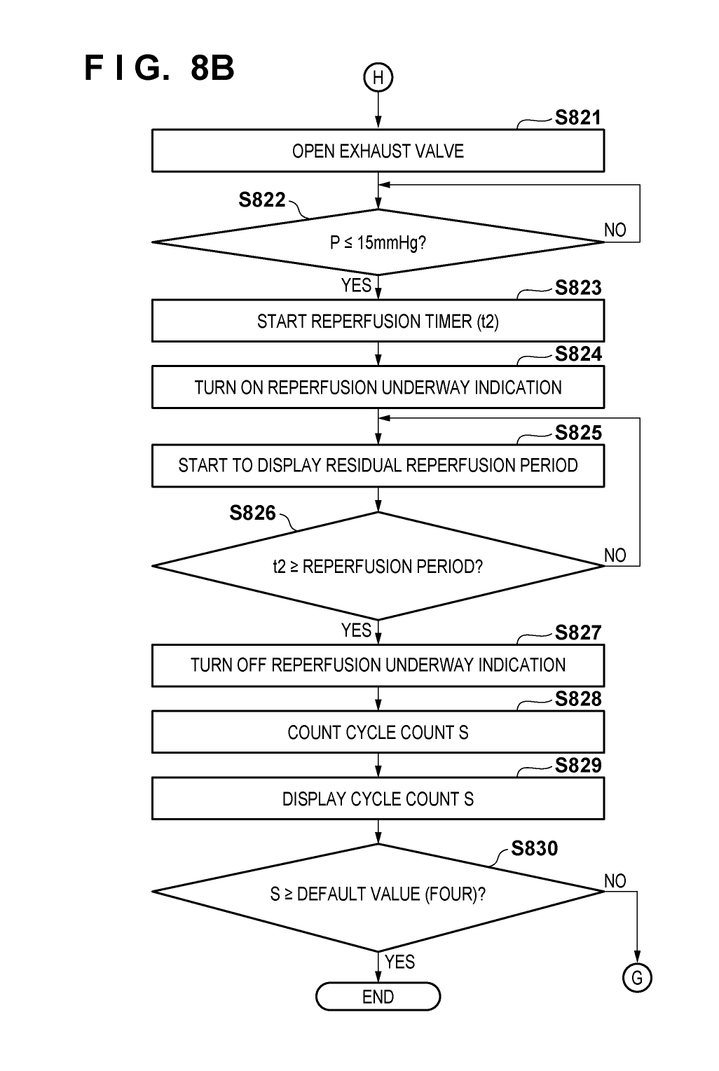

First of all, when the user presses the start/stop switch 102, the control unit 440 checks in step S101 in FIG. 5A whether the user has set the manual mode of the auto mode using the manual mode switch 108. If the user has set to the auto mode, the control unit 440 turns on the automatic mode indication 210 in step S102 and sets the device to operate in the automatic mode. If the user has set to the manual mode, the control unit 440 turns on the manual mode indication 211 in step S103 and sets the device in the manual mode. The process then advances to stop S803 (FIG. 8A) to set the device in the setting mode to allow the user to set a compression (occlusion) period, reperfusion period, and compression pressure with the setting switch 105 and the setting value increasing/decreasing switches 106 and 107.

FIGS. 5A and 5B show processing at the time of the first pressurization in the RICM processing automatic mode. When the user presses the start/stop switch 102 while the automatic mode is set, the control unit 440 turns on the automatic mode indication in step S102. In step S501, the control unit 440 performs various types of initialization. More specifically, the control unit 440 sets the initial pressure to zero and resets various types of timers (for example, a timer (t3) for measuring a pressurization time, a timer (t1) for measuring a compression period and a timer (t2) for measuring a reperfusion period). The control unit 440 also sets a repressurization count counter KT to zero. In this case, the control unit 440 sets the timer (t3) to the default value recorded in advance on the ROM of the control unit 440 based on the cuff size identified by the connection sensor 408 because there are differences in capacity among the cuff bladders. The setting value of the timer is automatically changed and set to a small default value for S size or a large default value for L size relative to the default value for M size. According to another method, the timer (t3) may be set to a fixed value, and the pressurization rate of the cuff may be set to a low rate for S size or a high rate for L size relative to the pressurization rate for M size. In step S502, the control unit 440 starts to receive the cuff pressure value output from the pressure detection unit 403, starts to display the value on the cuff pressure indication 207 of the display unit 103, and starts to receive a K-sound detection signal from the K-sound detection unit 409 via the A/D converter 441-1.

In step S503, the control unit 440 instructs the valve driving units 405a and 405b to fully close the constant-rate exhaust valve 404a and the exhaust valve 404b. In step S504, the control unit 440 instructs the pump driving unit 407 to drive the pump 406. Note that the pump driving unit 407 controls the rotational speed of the pump 406 by PWM control and can control the pressurization speed. When starting to drive the pump in step S504 (that is, in a pressurization process), the control unit 440 controls the driving operation with a duty ratio of 100% until, for example, the cuff pressure becomes the first default value (for example, 40 mmHg). This supplies air to the dead space in the cuff air bag at full speed and hence can quickly pressurize the cuff unit 301. Driving the pump 406 in this manner will start the first pressurization to set a compression state.

When starting to pressurize the cuff unit 301 with the pump 406 in step S504, the control unit 440 starts the pressurization timer (t3) in step S505. As described with reference to FIG. 4, the control unit 440 detects the size (one of S, M, and L sizes) of the connected cuff from an output value from the connection sensor 408. If the pressure value P of the cuff unit 301 has not reached the first default value (40 mmHg in this embodiment) within the first default time (for example, 15 sec) set in advance for each cuff size, the control unit 440 determines that an abnormality has occurred in the pump 406, and stops the processing by the corresponding cuff unit. That is, in step S506, the control unit 440 determines whether the pressurization time timer value (t3) is equal to or more than the first default time (15 sec in this embodiment). If “t3≧first default time”, the control unit 440 determines that an abnormality has occurred in the pump, and the process advances to the processing in step S531 and the subsequent steps. If “t3<first default time”, the process advances to step S507. In step S507, the control unit 440 determines whether the pressure value P detected by the pressure detection unit 403 is equal to or more than the first default value (for example, 40 mmHg). If the control unit 440 determines that “P≧first default value”, the process advances to step S508. Otherwise, the process returns to step S506.

In step S508, the control unit 440 keeps pressurizing the cuff unit 301 with the pump 406 and waits until the pressure value P becomes equal to or more than the second default value (for example, 180 mmHg in this embodiment). When the pressure value P becomes equal to or more than the second default value, the process advances to step S509, in which the control unit 440 instructs the pump driving unit 407 to stop the pump 406. In step S510, the control unit 440 instructs the valve driving unit 405a to open the constant-rate exhaust valve 404a to start to depressurize the cuff unit 301 at a constant rate (2 to 3 mmHg/sec). The control unit 440 monitors the detection of K-sound by the K-sound detection unit 409 while depressurizing the cuff unit 301 at the constant rate (step S511). When the control unit 440 detects K-sound, the process advances to step S513.

In step S513, the control unit 440 checks whether K-sound has been detected within a default time (1.5 sec) from the start of depressurization (detection of insufficiency of pressurization). Upon determining that K-sound has been detected within the default time, the control unit 440 determines that the cuff pressure is too low to measure a systolic blood pressure, that is, the pressurization is insufficient. If the pressurization is insufficient, the control unit 440 checks in step S519 whether the number of times pressurization insufficiency has occurred is two (checks whether the value of the repressurization count counter KT is 1). If the value of KT is equal to or more than 1, the control unit 440 determines that it is not possible to perform automatic mode operation using K-sound. The process therefore advances to step S801 to execute processing in the manual mode. In step S801, the control unit 440 instructs the valve driving unit 405b to open the exhaust valve 404b, and stops the operation. In contrast, if the value of KT is 0, for example, the control unit 440 changes the second default value to 220 mmHg (step S520), and counts up KT (step S521). The control unit 440 then closes the exhaust valves 404a and 404b again (step S522) and starts the pump (step S523). The process then returns to step S508.

Note that in this embodiment, if K-sound corresponding to two consecutive pulsations is detected, the control unit 440 determines that K-sound is detected (step S511). If the pressure value P becomes smaller than the third default value (for example, 70 mmHg: the lower limit value of normal systolic blood pressures) without detection of any K-sound (YES in step S512), the control unit 440 determines that the pump is normal but it is not possible to perform control using K-sound, and the process advances to step S801 in FIG. 8A. In step S801 and the subsequent steps, the control unit 440 changes over to the manual mode to allow the user to manually set a pressure value at the time of compression, and executes RICM processing without using any K-sound.

If K-sound corresponding to two consecutive pulsations is detected in step S511 and the control unit 440 determines in step S513 that no pressurization insufficiency has occurred, the control unit 440 sets the cuff pressure value P corresponding to the first detection of K-sound to SBP in step S514. With this processing, a pressure value corresponding to the systolic blood pressure value of the patient is automatically set to SBP. The control unit 440 then instructs the valve driving units 405a an 405b to close the exhaust valves 404a and 404b (step S515), and instructs the pump driving unit 407 to start the pump (step S516). In this manner, the control unit 440 pressurizes the cuff unit 301 until the pressure value P becomes SBP+20 mmHg, and then stops the pump 406 (steps S517 and S518), and shifts to the processing in a compression period (the processing in step S601 and the subsequent steps). Although the above description has presented 20 mmHg as a predetermined value to be added to SBP, the present invention is not limited to this.

According to the pressurization processing described above, the device automatically measures the systolic blood pressure value of a patient, and in an ischemic period, the cuff unit 301 executes compression with a pressure value higher than the measured systolic blood pressure value by a predetermined pressure (by 20 mmHg in this case). That is, according to this embodiment, the device automatically sets a proper pressure value (systolic blood pressure+20 mmHg) for the cuff unit 301 in accordance with the blood pressure state of a patient, and can reliably set an ischemic state near the systolic blood pressure of the patient.

Upon determining that an abnormality has occurred in the pump (YES in step S506), the control unit 440 instructs the pump driving unit 407 in step S531 to immediately stop driving the pump 406. In step S532, the control unit 440 instructs the valve driving unit 405a to fully open the constant-rate exhaust valve 404a, and also instructs the valve driving unit 405b to fully open the exhaust valve 404b. In addition, in step S533, the control unit 440 blinks the pump abnormality indication 208. In this case, if the user has selected the display associated with another cuff system with the cuff changeover switch 104, the control unit 440 automatically changes over to the display associated with the corresponding cuff system to notify the user of the pump abnormality.

As described above, when starting to pressurize the cuff unit 301 by starting to drive the pump 406, the control unit 440 monitors the cuff pressure in the cuff unit 301. If the cuff pressure has not reached the first default value within the first default time, the control unit 440 determines the occurrence of connection failure at the connector on the proximal end of the tube 111, air leakage, or pump function abnormality, stops driving the pump 406, and outputs a warning to the user. This allows the user to detect an abnormality in the pump or K-sound detection system early in the pressurization process. In addition, if no K-sound is detected or the disappearance of K-sound cannot be detected in spite of the fact that a sufficient pressure value is obtained within the first default time, the device operates in the manual mode of performing operation using a manually set compression pressure value without performing pressure control using K-sound. Processing in this mode will be described later with reference to the flowcharts of FIGS. 8A and 8B.

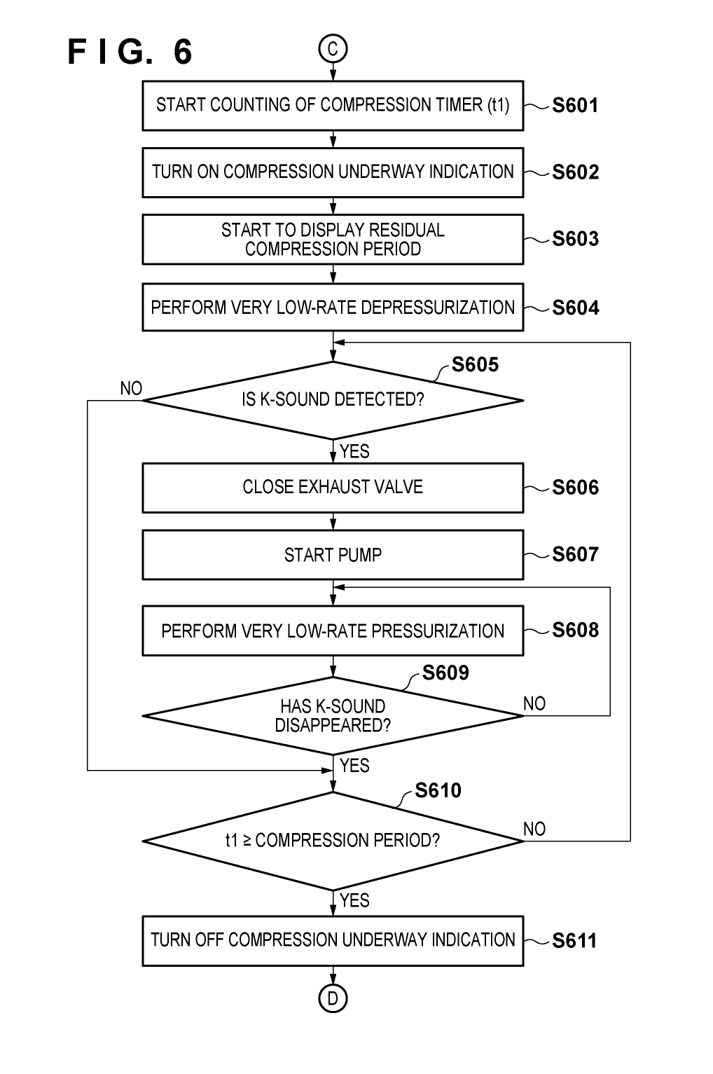

FIG. 6 is a flowchart for explaining processing in a compression period. First of all, in step S601, the control unit 440 starts the compression timer (t1) for measuring an compression period. In step S602, the control unit 440 turns on the compression underway indication 203 of the display unit 103 to notify the user that compression is underway. In step S603, the control unit 440 starts displaying the residual period until the completion of compression on the residual period display area 205 of the display unit 103 based on the timer value (t1) of the compression timer. Note that the device calculates the residual time by subtracting the timer value (t1) of the compression timer from a predetermined compression period or the compression period set by the user by using the operation input unit 109.

In step S604, the control unit 440 instructs the valve driving unit 405a to start exhausting air at a very low rate (0.1 to 0.5 mmHg/sec) using the constant-rate exhaust valve 404a. In step S605, the control unit 440 performs K-sound detection. Upon detecting no K-sound, the control unit 440 detects in step S610 whether the timer value (t1) is equal to more than a set compression period. Upon detecting K-sound in step S605, the control unit 440 instructs the valve driving unit 405a to close the constant-rate exhaust valve 404a (step S606), and starts the pump (step S607) to perform very low-rate pressurization at a pressurization rate of about 2 to 3 mmHg/sec (step S608). At this time, noise from the pump is eliminated by the silencer 406a and hence cause no trouble in K-sound detection. In step S609, the control unit 440 checks whether K-sound has disappeared. As a K-sound disappearance detection method, there is available a method of determining the disappearance of K-sound if, for example, no K-sound is detected in 1.5 sec consecutively twice, which correspond to one period of a normal pulse rate, that is, 40 pulses/min, that is, a total of 3 sec. Obviously, the present invention is not limited to this determination method. For example, the control unit 440 may calculate an average period of K-sound detected up to now, and determines the disappearance of K-sound if detecting no K-sound consecutively twice in the average period.

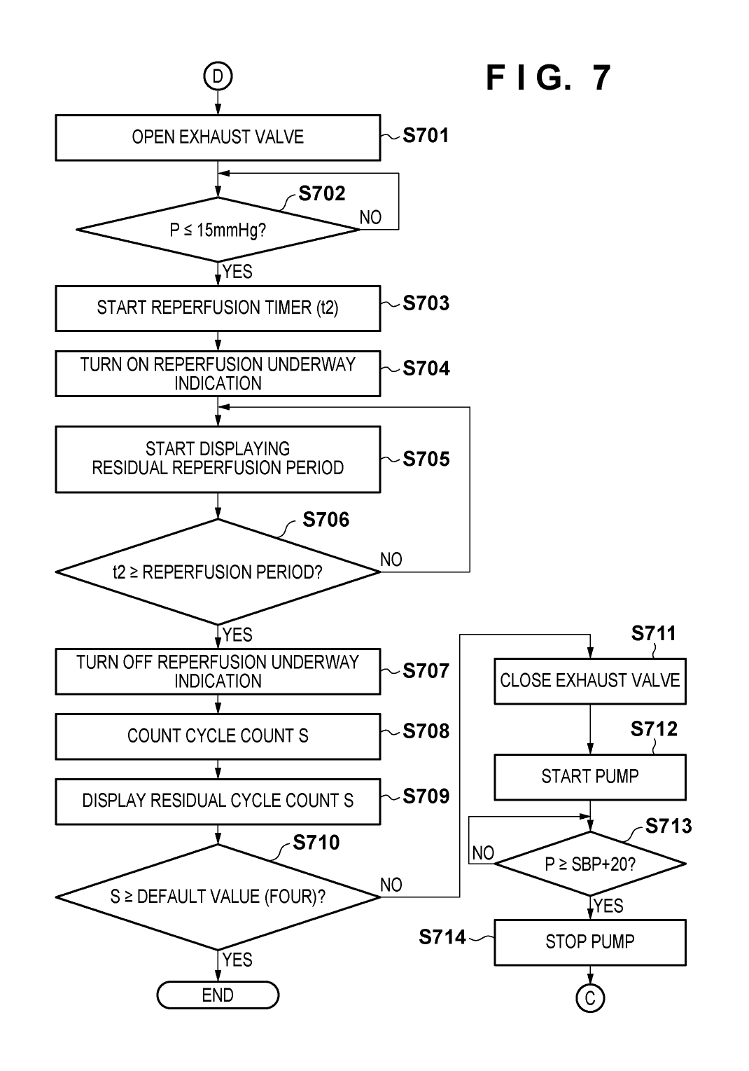

If the control unit 440 does not detect the disappearance of K-sound, the process returns to step S608 to continue very low-rate pressurization and detection of the disappearance of K-sound. If the control unit 440 detects the disappearance of K-sound, the process returns to step S610 to check whether a set compression period has elapsed. In the above manner, the control unit 440 monitors K-sound and the pressure value P, and controls the pump driving unit 407 and the valve driving units 405 so as to maintain the compression pressure at (SBP+10 mmHg or less). When the timer value (t1) of the compression timer becomes equal to or more than the compression period (step S610), the process advances to step S611, in which the control unit 440 turns off the compression underway indication 203. The process then advances to step S701 in FIG. 7 to start processing in a reperfusion period.

In this manner, the control unit 440 performs compression while maintaining the cuff pressure value near a value slightly larger than the systolic blood pressure value of a patient during the compression period, and hence reduces the burden of treatment on the patient.

FIG. 7 is a flowchart showing processing in a perfusion period. In step S701, the control unit 440 instructs the valve driving units 405 to fully open the constant-rate exhaust valve 404a and the exhaust valve 404b. As described above, when shifting from a compression state to a reperfusion state after the lapse of a compression period, the control unit 440 fully opens the exhaust valve 404b as well as the constant-rate exhaust valve 404a. This makes it possible to depressurize the cuff unit 301 at a high rate.

In step S702, the control unit 440 determines whether the cuff pressure value output from the pressure detection unit 403 is equal to or less than 15 mmHg. If the control unit 440 determines in step S702 that the cuff pressure value is neither equal to nor less than 15 mmHg, the process waits until the cuff pressure value becomes equal to or less than 15 mmHg.

If the control unit 440 determines in step S702 that the cuff pressure value is equal to or less than 15 mmHg, the process advances to step S703. In step S703, the control unit 440 starts the reperfusion timer (t2) for measuring a reperfusion period. In step S704, the control unit 440 turns on the reperfusion underway indication 204 to notify the user that reperfusion is underway. In step S705, the control unit 440 starts turning on the residual period indication on the residual period display area 205 of the display unit 103 until reperfusion is complete based on the timer value (t2) of the reperfusion timer. Note that the residual period is calculated by subtracting the timer value (t2) of the reperfusion timer from a predetermined reperfusion period (for example, 5 min).

In step S706, the control unit 440 determines whether the timer value (t2) of the reperfusion timer is equal to or more than a predetermined reperfusion time or the reperfusion time (for example, 5 min) set by the operation input unit 109. If the control unit 440 determines in step S706 that the timer value (t2) has not reached the set reperfusion period (for example, min), the process returns to step S705 to calculate and display a residual reperfusion period. With this operation, the device executes perfusion for the set reperfusion period.

Upon determining in step S706 that the timer value (t2) is equal to or more than the reperfusion period (for example, 5 min) set by the operation input unit 109, the control unit 440 turns off the reperfusion underway indication 204 in step S707. In step S708, the control unit 440 increments a cycle count S (assume that 0 as an initial value has been input to the count value of the cycle count). The process further advances to step S709 in which the control unit 440 displays the residual cycle count in the residual cycle count display area 206 of the display unit 103. Note that a residual cycle count is calculated by subtracting the currently counted cycle count S from a predetermined count or the count (four cycles in this embodiment) set by the operation input unit 109.

If the control unit 440 determines in step S710 that the counted cycle count S is less than the above default value (four cycles in this embodiment), the process advances to step S711 to set an ischemic state again. In contrast, upon determining in step S710 that the cycle count S is equal to or more than the default value, the control unit 440 terminates the RICM processing.

In step S711, the control unit 440 instructs the valve driving units 405a and 405b to fully close the constant-rate exhaust valve 404a and the exhaust valve 404b. In step S712, the control unit 440 instructs the pump driving unit 407 to drive the pump 406 and make the cuff unit 301 start to pressurize the region to be compressed. Note that the pressurization speed at this time is equal to the pressurization speed in step S504. When the pressure value P detected by the pressure detection unit 403 becomes equal to or more than the value obtained by adding 20 mmHg to SBP set in step S514, the control unit 440 stops the pump. The process then returns to step S601 to perform processing in a compression period (steps S713 and S714).

The above processing is the processing of maintaining a compression pressure in a compression period at a pressure suitable for a patient by using K-sound. In contrast, upon determining that it is not possible to set a compression pressure by using K-sound (blood pressure measurement result) (YES in step S512 or S519), the control unit 440 executes RICM processing upon manually setting a compression pressure. FIGS. 8A and 8B are flowcharts for explaining such processing. First of all, in step S801, the control unit 440 instructs the valve driving units 405a and 405b to fully open the constant-rate exhaust valve 404a and the exhaust valve 404b to stop pressurization. In step S802, the control unit 440 turns on the K-sound detection abnormality indication 209 to display an alarm and notify the user of inability to use K-sound. In step S803, the control unit 440 makes the user input a compression pressure (a manually set compression pressure value will be referred to as SBPX hereinafter) by using the setting value increasing/decreasing switches 106 and 107. At this time, if the display unit 103 displays another cuff system or an item other than the compression pressure item is selected as a setting item for the setting value increasing/decreasing switches 106 and 107, the device may automatically change over to an input state for a compression pressure for the corresponding cuff system.

In step S811, the control unit 440 instructs the valve driving units 405a and 405b to close the constant-rate exhaust valve 404a and the exhaust valve 404b, respectively. In step S812, the control unit 440 instructs the pump driving unit 407 to start the pump 406, and waits until the pressure value P of the cuff unit 301 detected by the pressure detection unit 403 becomes equal to or more than SBPX input in step S803 (step S813). Note that the pressurization speed at this time is equal to the speed of pressurization started in step S504. When the pressure value P becomes equal to or more than SBPX, the process advances from step S813 to step S814. The control unit 440 instructs the pump driving unit 407 to stop the pump 406.

The control unit 440 starts the compression timer (t1) in step S815, and turns on the compression underway indication 203 in step S816. In step S817, the control unit 440 starts to display a residual period until the completion of compression in the residual period display area 205 of the display unit 103 based on the timer value (t1) of the compression timer. Note that this display processing is the same as that in step S602. In step S818, the control unit 440 waits until the timer value (t1) becomes equal to or more than the compression period, and maintains the compression state. Note that in this period, the control unit 440 may monitor the cuff pressure value P to control the pump driving unit 407 and the valve driving units 405a and 405b so as to maintain the pressure value near the compression pressure (SBPX).

When the timer value (t1) of the compression timer becomes equal to or more than the compression period, the process advances to step S819, in which the control unit 440 turns off the compression underway indication 203. The process advances to step S821 to start processing in a reperfusion period. The processing in the reperfusion period shown in steps S821 to S830 is the same processing in the reperfusion period described with reference to steps S701 to S710 in FIG. 7. If the cycle count S is equal to or more than the default value (four in this embodiment) in step S830, the control unit 440 terminates this processing. Otherwise, the process returns to step S811 to shift to processing in a compression period in the next cycle.

### 6. Description of Pressure Control

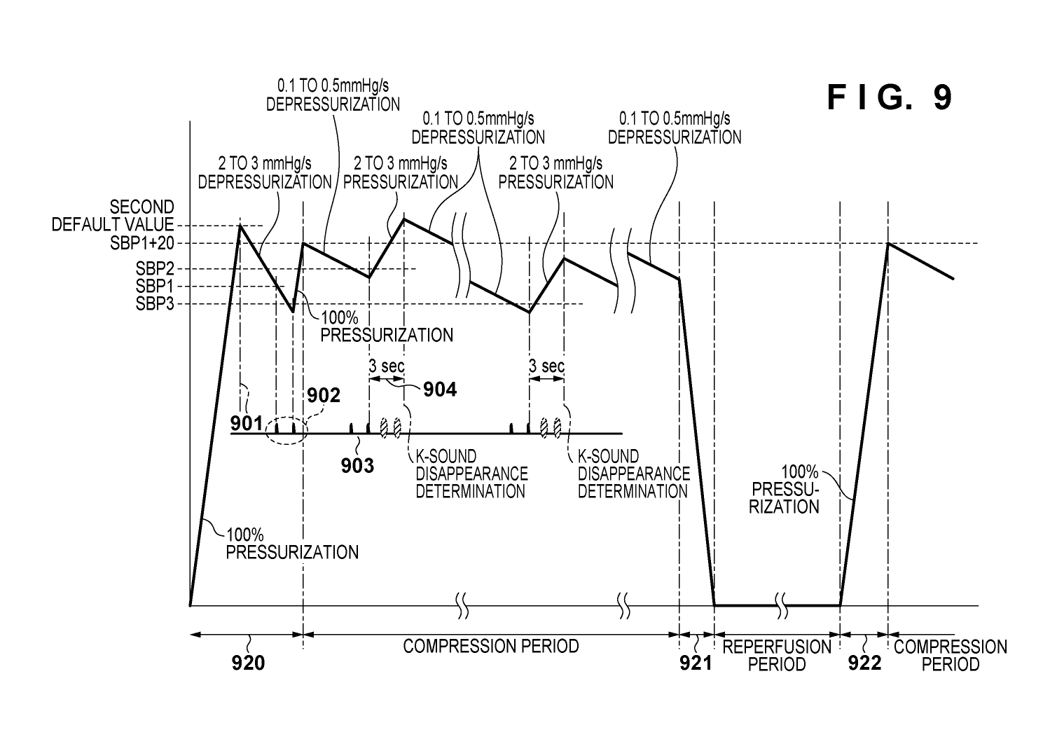

Pressure control by the limb compression device 100 at the time of RICM processing using K-sound (pressure measurement result) described with reference to the flowcharts of FIGS. 5A, 5B, 6, and 7 will be described next. FIG. 9 is a graph for explaining pressure control by the limb compression device 100 at the time of RICM processing.

As shown in FIG. 9, the device increases the cuff pressure of the cuff unit 301 to the second default value by the first pressurization (901 and step S508). Thereafter, the device depressurizes the cuff unit 301 at 2 to 3 mmHg/s and monitors K-sound detection by the K-sound detection unit 409. Upon detecting K-sound (902 and step S511), the device holds the pressure value P at this time point as SBP1 (Systolic Blood Pressure) and increases the pressure of the cuff unit 301 once to SBP1+20 mmHg. Subsequently, the device monitors K-sound while performing very low-rate depressurization (0.1 to 0.5 mmHg/sec). If K-sound corresponding to two consecutive pulses is detected (903), the control unit 440 determines that the cuff pressure has become too low to maintain compression (occlusion by cuff) due to variations in blood pressure, and observes K-sound while pressurizing the cuff at a pressurization rate of 2 to 3 mmHg/sec (steps S605 to S609). The control unit 440 then detects that no K-sound is continuously detected for 3 sec, and detects that a proper compression state is set, thereby restoring the compression state (904). Subsequently, the control unit 440 instructs the valve driving unit 405a to open the constant-rate exhaust valve 404a to start very low-rate depressurization, and monitors K-sound. Repeating the above control can set, as an ischemic pressure, a pressure (corresponding to two pulsations; a maximum pressure of about 10 mmHg) slightly higher than SBP while measuring SBP by using K-sound. FIG. 9 shows cuff pressure control changes in a case in which the systolic blood pressure has changed to a pressure (SBP2) higher than the initial pressure (SBP1) and in a case in which the systolic blood pressure has changed to a lower pressure (SBP3). In case where the systolic blood pressure has changed high and a pulse rate of a patient is 60 pulses/min, the device sets, as an compression pressure, the cuff pressure which is increased to a pressure higher than the systolic blood pressure by about 6 to 9 mmHg. The device may set, as a compression pressure, the cuff pressure increased to the pressure obtained by further adding an arbitrary numerical value in consideration of the stability of compression.

Note that since an optimal compression period and reperfusion period are set in accordance with the state of a patient, it is preferable to minimize the time for pressurization or depressurization (920, 921, 922) between a compression period and a reperfusion state. That is, a system for pressure control during state transition between a compression period state and a reperfusion state is preferably configured as a system with high responsibility.

It is preferable to optimize a compression pressure, while maintaining a compression state, in accordance with the state of a patient, so as to minimize the burden on the patient. It is also preferable to perform control to maintain a proper compression pressure in accordance with the patient during a compression period. Under such circumstances, the limb compression device 100 according to this embodiment sets a compression pressure to a pressure value (almost SBP+10 mmHg or less) slightly higher than the systolic (the maximum) blood pressure of a patient. FIG. 9 shows a state in which the device monitors the pressure value of the cuff unit 301 during a compression period and performs control so as to make the pressure value of the cuff unit 301 fall within a predetermined range of compression pressure values.

Obviously, from the above description, the limb compression device 100 according to this embodiment can maintain and manage a compression state while making settings so as to minimize burden on a patient, when executing RICM processing, in consideration of variations in blood pressure (systolic blood pressure value) of the patient, and can implement proper limb compressing operation in accordance with the settings.

That is, it is possible to provide a limb compression device which can maintain and manage a compression state and implement limb compressing operation so as to minimize burden on a patient.

### Other Embodiments

Note that the first embodiment prepares the four cuff units for the right lower limb, left lower limb, right upper arm, and left upper arm, and is configured to independently perform pressure control on them. However, the present invention is not limited to this. For example, the device may include four or more cuff units or less than four cuff units and non-independently perform pressure control.

In addition, the first embodiment is configured to separately provide the exhaust valve to be used for very low-rate depressurization in a compression state and the exhaust valve to be used in a depressurization process for shifting from a compression state to a reperfusion state. However, the present invention is not limited to this. The device may be configured to use one exhaust valve by changing its opening degree in accordance with a compression state and a depressurization process.

In addition, the first embodiment described above performs completely independent control for each cuff system. However, the present invention is not limited to this. For example, if no pulsation (K-sound in this embodiment) can be detected on the peripheral side relative to a compressed region, it is possible to use SBP obtained by another cuff system of the plurality of cuff systems. In this case, it is preferable to use SBP obtained by a cuff system at a region which may exhibit the systolic blood pressure value similar to the blood pressure value. For example, the blood pressure value in the right lower limb may be used as the blood pressure in the left lower limb. Alternatively, coefficients to be applied among the respective regions may be set in advance to use a blood pressure value at another region, like using, for the right upper limb, the value obtained by multiplying a blood pressure value at the right lower limb by a coefficient. Therefore, the device may be configured to provide a K-sound detection unit for only a cuff system for the left upper limb and decide SBP for the left and right lower limbs and the right upper limb by applying coefficients to the measurement result. Note however that a cuff system which cannot detect K-sound cannot detect variations in systolic blood pressure in a compression period, and hence is configured to perform compression by maintaining a compression pressure value based on the systolic blood pressure decided in the above manner.

The present invention is not limited to the above embodiments and various changes and modifications can be made within the spirit and scope of the present invention. Therefore, to apprise the public of the scope of the present invention, the following claims are made.

What is claimed is:

1. A limb compression device to which a cuff unit wound around a limb of a patient is connected and which controls compression and release of the limb by controlling air supply and exhaust of the cuff unit, said limb compression device comprising:
a compression unit configured to compress the limb over a predetermined compression period by controlling air supply and exhaust of the cuff unit;
a release unit configured to release the compression of the limb over a predetermined reperfusion period by controlling exhaust of the cuff unit;
a control unit configured to repeat the compression period by said compression unit and the reperfusion period by said release unit by a predetermined number of times; and
a detection unit configured to detect pulsation on a peripheral side relative to a region compressed by the cuff unit,
wherein said compression unit
performs pressurization using the cuff unit up to a compression pressure value based on a systolic blood pressure of the patient measured by detection of pulsation using said detection unit, and
repeats, in the compression period, depressurization of the cuff unit at a low rate until pulsation is detected by said detection unit and pressurization of the cuff unit after the depressurization so as to eliminate pulsation.

2. The device according to claim 1, wherein when performing first pressurization for a first compression period, said compression unit performs compression up to a predetermined pressure value by using said cuff unit and then decides a compression pressure value based on a systolic blood pressure measured by depressurizing said cuff unit, and
when performing first pressurization for second and subsequent compression periods, performs compression by using said cuff unit up to a compression pressure value decided by first pressurization for the first compression period.

3. The device according to claim 1, wherein the compression pressure value is a value obtained by adding a predetermined value to a measured systolic blood pressure.

4. The device according to claim 1, wherein said device comprises a plurality of cuff systems each including said cuff unit, said compression unit, said release unit, said control unit, and said detection unit, each of said plurality of cuff systems independently executing iterations of the compression period and the reperfusion period.

5. The device according to claim 1, further comprising a setting unit configured to make a user set a compression pressure value when no systolic blood pressure of the patient is measured,
wherein said compression unit maintains a cuff pressure of said cuff unit at a compression pressure value set by said setting unit in the compression period.

6. The device according to claim 4, further comprising a setting unit configured to set, when no systolic blood pressure of the patient is measured, an compression pressure value by using a systolic blood pressure measured by another cuff system of said plurality of cuff systems,
wherein said compression means maintains a cuff pressure of said cuff unit at the compression pressure value set by said setting unit in the compression period.

7. A method of controlling a limb compression device to which a cuff unit wound around a limb of a patient is connected and which controls compression and release of the limb by controlling air supply and exhaust of the cuff unit, said method comprising:
a compression step of compressing the limb over a predetermined compression period by controlling air supply and exhaust of the cuff unit;
a release step of releasing compression of the limb over a predetermined perfusion period by controlling exhaust of the cuff unit;
a control step of repeating the compression period in the compression step and the reperfusion period in the release step by a predetermined number of times; and
a detection step of detecting pulsation on a peripheral side relative to the region compressed by the cuff unit,
wherein in the compression step, pressurization using the cuff unit is performed up to a compression pressure value based on a systolic blood pressure of the patient measured by detection of pulsation in the detection step, and
depressurization of the cuff unit at a low rate until pulsation is detected in the detection step and pressurization of the cuff unit after the depressurization so as to eliminate pulsation are repeated in the compression period.
